(19) United States
(12) Patent Application Publication
(10) Pub. No.: US 2007/0099259 A1
(43) Pub. Date: May 3, 2007

(54) ELECTROCHEMICAL ASSAY FOR THE IDENTIFICATION OF MICROORGANISMS

(75) Inventors: Susan R. MIKKELSEN, Kitchener, ON (CA); Peter ERTL, Guelph, ON (CA); Doug SPARKES, Kitchener, ON (CA); Liam O'HAGAN, Walkerton, ON (CA); Thomas MANN, Hanover, ON (CA); Paul ULLRICH, Kitchener, ON (CA)

Correspondence Address: BORDEN LADNER GERVAIS LLP, WORLD EXCHANGE PLAZA, 100 QUEEN STREET SUITE 1100, OTTAWA, ON, K1P 1J9, CA

(73) Assignee: RAPID LABORATORY MICROSYSTEMS INC., Kitchener, ON (CA)

(21) Appl. No.: 10/596,460

(22) PCT Filed: Dec. 3, 2004
(86) PCT No.: PCT/CA04/02076

§ 371 Date: Jun. 14, 2006

Related U.S. Application Data
(60) Provisional application No. 60/529,403, filed on Dec. 15, 2003.

(51) Int. Cl. C12Q 1/04

(52) U.S. Cl. 435/34

(57) ABSTRACT

A method for the phenotypic identification of microorganisms is provided. The method is based on the evaluation of the effects of various compounds (effectors) on the respiratory cycle activity of microorganisms. Measurements are based upon the ability of the microorganism to transport electrons to an external chemical oxidant (a mediator) that is added to the microorganism sample. The mediator interacts with the terminal components of the respiratory pathway and the extent of its consumption is related to the ability of the microorganism to respire. The consumed mediator is subsequently measured electrochemically. Electrochemical signals which are generated in the presence or absence of an effector can be used to generate a signal pattern that is unique to an organism and can be used for identification.

### FIELD OF THE INVENTION

The present invention relates to assays for identifying microorganisms. More particularly, the present invention relates to testing prokaryotic and eukaryotic microorganisms in a multitest format for the rapid identification and characterization of cultures important to clinical, agricultural and environmental testing.

### BACKGROUND OF THE INVENTION

Many species of microorganisms are harmful to humans or animals, and for this reason, the correct identification of pathogens that cause disease is of great concern. Microbial diseases constitute the major cause of death in many developing countries of the world. A growing number of microbial pathogens have been identified as important food- and waterborne pathogens.

Traditional microbial identification methods involve a pre-enrichment step and a selective enrichment step, followed by morphologic examination, biochemical screening, growth characterization, serotyping and serological confirmation. The enrichment steps consist of growth on selective media. Morphologic examination includes observation of colony characteristics and pigmentation, microscopic examination after staining (e.g. Gram stain, acid-fast stain), examination for motility and arrangement of flagella, examination for the presence of spores, capsules and inclusion bodies, and determination of the shape of the organism. Biochemical screening consists of examination for the presence or absence of constitutive enzymes or metabolic pathways in the enriched microbial culture; some biochemical tests are used generally (e.g. oxidase, nitrate reduction, amino acid-degrading enzymes, fermentation and utilization of carbohydrates), while others are restricted (e.g. coagulast test for Staphylococcus spp.). Growth characteristics determine whether an organism is aerobic, anaerobic, facultative or microaerophilic, the specific temperature and pH conditions required for growth, the nutrient requirements, and resistance to antibiotics. Serotyping uses antibodies to determine the type of O antigen (cell wall), H antigen (flagellar) and K antigen (capsular) present on the organism and allows both species and strain identification. Serological confirmation consists of in vitro reaction of patient serum (containing antibodies) with the isolated organism.

Modern microbial identification methods are classified as either genotypic or phenotypic methods. Genotypic methods include all forms of analysis of microbial DNA and RNA. These methods detect genetic material present in the microorganism that, if expressed, would allow the synthesis of biochemical pathway components (e.g. for metabolism, stress response factors, antibiotic resistance mechanisms).

Genotypic methods do not provide an assessment of active pathways present in a given microorganism culture at a given time. Many genotypic methods have been proposed for microbial identification and antibiotic resistance testing, including US Patent Applications 20020187490 (Tiedje et al.), 20030049636 (Bergeron et al.), 20030124545 (Rothman et al.), 20030180733 (Bergeron et al.), and 20040023208 (Haydock et al.).

Phenotypic identification methods assess the portion of the microbial genome that is currently expressed in a given microbial culture. These methods include traditional methods based on the observation of growth under varied conditions as well as biochemical screening for the presence of metabolic pathways. Many inventions describe methods and apparati for the detection of growth and for biochemical screening in multitest formats including U.S. Pat. No. 3,957,586 (Babson et al.), U.S. Pat. No. 4,118,280 (Charles et al.), U.S. Pat. No. 4,129,483 (Bochner), U.S. Pat. No. 4,235,964 (Bochner), U.S. Pat. No. 4,456,380 (Kondo et al.), U.S. Pat. No. 4,603,108 (Bascomb), U.S. Pat. No. 4,604,351 (Amaral), U.S. Pat. No. 4,724,215 (Farber), U.S. Pat. No. 4,856,073 (Farber et al.), U.S. Pat. No. 5,089,395 (Snyder et al.), U.S. Pat. No. 5,164,301 (Thompson et al.), U.S. Pat. No. 5,223,402 (Abbas et al.), U.S. Pat. No. 5,340,747 (Eden), U.S. Pat. No. 5,843,699 (Strenkoski et al.), U.S. Pat. No. 5,888,760 Godsey et al.), U.S. Pat. No. 6,372,485 (Clark et al.), U.S. Pat. No. 6,387,651 (Bochner et al.), U.S. Pat. No. 6,436,631 (Bochner et al.), U.S. Pat. No. 6,555,322 (James et al.), and U.S. Pat. No. 6,611,765 (Boeufgras et al.), as well as US Patent Applications 20020064867 (Clark et al.), 20030032171 (Gemmell et al.), 20030162164 (Bochner et al.) and 20040009572 (Felice et al.) and World Patent Application WO2004050675 (Taintor). Electrochemical measurements on microorganisms are described in U.S. Pat. No. 6,391,577 (Mikkelsen et al.) and Ertl, P. et al. (2000), Analytical Chemistry 72:4957-64.

Another phenotypic method has been described in which cell membrane potential changes are measured using a fluorescent dye after exposure of a culture to a bioactive chemical or biochemical in U.S. Pat. No. 4,343,782 (Shapiro). A database method for microbial fingerprinting by pattern recognition of pyrolysis mass spectrometric data has been described in US Patent Application 20020138210 (Wilkes et al.). Another database method for microbial fingerprinting has been described in US Patent Application 20030097059 (Sorrell et al.) for matching of microbial magnetic resonance data patterns. A third database method involves capture of microorganisms on surfaces modified with specific compounds designed to recognize and bind to functional groups on the surfaces of microorganisms; the binding pattern for a given organism is compared to a database of binding patterns for different organisms as described in U.S. Pat. No. 6,780,602 (Lloyd et al.).

Generally, no single test provides a definitive identification of an unknown microorganism. Further, currently available tests are time-consuming and often unreliable. There is a need for a rapid and effective method of identifying unknown microorganisms.

### SUMMARY OF THE INVENTION

It is an object of the present invention to obviate or mitigate at least one disadvantage of previous microorganism assays.

In one aspect of the present invention there is provided a method of identifying a microorganism comprising the steps of: obtaining a test sample of an unknown microorganism; adding a mediator or mediator mixture to the test sample in the presence of an effector; assessing variation in respiration rate of the microorganism over a predetermined time period; and comparing the variation in the respiration rate of the microorganism with the variation in respiration rates of known microorganisms exposed to the effector, thereby identifying the unknown microorganism in the test sample.

In another aspect of the present invention there is provided a method of differentiating Gram-positive and Gram-negative bacteria comprising the steps of: obtaining a test sample of a microorganism; adding a lipid-soluble redox-mediator to one portion of the test sample; assessing variations of respiration rate of the microorganism over a predetermined time period; and comparing the respiration rate of this portion of the test sample with the respiration rate of another portion of the test sample not exposed to the lipid-soluble redox mediator.

Surprisingly, it has been found that respiratory rate measurements taken after mixture of a test sample with an effector compound can be used for the identification and differentiation of microorganisms. The method for assessing cell responses to external stimuli comprises adding a suitable mediator or mediator mixture to a sample of the microorganism in the presence of effectors, and assessing variation of the microorganism's respiration rate over time by electrochemical measurement of mediator consumption resulting from microorganism respiration. This is compared with variation of the respiration rate to other microorganism samples (stock organisms) exposed to the same effectors.

### BRIEF DESCRIPTION OF THE FIGURES

Embodiments of the present invention will now be described, by way of example only, with reference to the attached Figures, wherein:

FIG. 1 shows plots of consumed charge against time for Enterobacter aerogenes ATCC 13048e samples from Example 1. The potential difference between the two platinum electrodes (area=0.017 cm.sup.2) is fixed at −100 mV. Traces shown are duplicate measurements and were obtained in the presence of 10 mM (a) glucose, (b) lactic acid, (c) pyruvic acid, (d) arginine and in (e) potassium nitrate.

FIG. 2 shows scores plots from Principal Component Analysis of respiratory activity data obtained using 22 different effector compounds in order to distinguish 10 microbial strains.

FIG. 3 shows scores plots from Principal Component Analysis of respiratory activity data obtained using 16 effector compounds in order to distinguish 10 microbial strains. The scores plot was generated using variances found in PC 2 and PC 4.

FIG. 4 shows a 3-dimensional scores plot from Principal Component Analysis of respiratory activity data obtained using 9 effector compounds in order to distinguish 10 microbial strains.

### DETAILED DESCRIPTION OF THE INVENTION

Generally, the present invention provides a rapid new method to be used for the differentiation and identification of various microorganisms, or for the differentiation between Gram-positive and Gram-negative bacteria, or for the determination of the presence and potency of effector compounds in samples using microorganisms of known identity. More particularly, the present invention provides a method of identifying a microorganism comprising the steps of: obtaining a test sample of an unknown microorganism; adding a mediator or mediator mixture to the test sample in the presence of an effector; assessing variation in respiration rate of the microorganism over a predetermined time period; and comparing the variation in the respiration rate of the microorganism with the variation in respiration rates of known microorganisms exposed to the effector, thereby identifying the unknown microorganism in the test sample.

The present invention is based on the measurement of respiration (breathing) rates in microorganisms. Under natural conditions, microorganisms consume molecular oxygen (if they are aerobic) or another oxidant (if they are anaerobic). With aerobic organisms, oxygen is converted to water by proteins present in the membranes of the organisms. The oxygen is transformed by a process known as reduction, in which four protons and four electrons are supplied from the interior of the cell, causing the conversion of oxygen to water. Similar processes occur in anaerobic organisms, causing the reduction of oxidants such as nitrate or sulfate. These reduction processes can be made to occur with other oxidants, or mediators, that can also accept electrons from cells and transfer them to a conducting, amperometric electrode, to generate a current that is measured. The electrodes and measuring equipment used in the invention are commercially-available and well-characterized.

The present invention provides a rapid method for evaluating the effects of various compounds on microorganisms based upon the microorganism's ability to transport electrons to an external chemical oxidant (a mediator) that is added to the microorganism sample. The mediator interacts with the terminal components of the respiratory pathway and the extent of its consumption is related to the ability of the microorganism to respire. However, under the assay conditions described herein the extent of mediator consumption is different from the microorganisms' ability to consume oxygen or another native oxidant, due to the addition of metabolizable compounds, or other compounds that affect respiratory activity, to the assay mixtures. The consumed mediator is subsequently measured electrochemically (biamperometrically or coulometrically) at the working electrode of a two-electrode electrochemical cell consisting of two polarizable platinum electrodes of approximately equal surface area. This measurement can also be made using two- or three-electrode cells in which one electrode is a nonpolarizable reference electrode; a sufficiently oxidizing potential is applied to the working electrode (with respect to the reference) to reoxidize the microbially-reduced mediator. The electrochemical signals (current or charge measured as a function of time) obtained with microorganism suspensions incubated in the presence of various effector compounds are significantly different. These signal differences can be used to generate a pattern that differentiates and identifies microorganisms.

The method of the present invention may further comprise a sample preparation step, in which the cell culture or suspension of microorganisms is combined with a solution of the proposed effectors and incubated for a fixed time, a second step in which a mediator or mediator mixture is added, and a third step in which a biamperometric measurement is made at fixed applied potential, using standard, commercially available electrochemical instrumentation (a potentiostat) and a 2-electrode electrochemical cell. In embodiments of the present invention, the first two steps may be combined into one step. In the absence of effectors, the mediator is converted by the microorganism from the oxidized to the reduced form at a rate that is characteristic of the organism and the concentrations of organism and mediator in the sample. During the measurement step, the reduced mediator is reconverted to the oxidized form at the working electrode by applying a voltage difference between the two electrodes, and the magnitude of the measured current is proportional to reduced mediator concentration in the sample.

In the presence of effectors during the incubation and measurement steps, the rate of mediator reduction, and the resulting measured signals, is significantly different for each effector compound and organism. Depending on the chemical nature of these effectors and interaction with the target organisms, obtained respiratory activities can reach values between 10 to 600% if compared to control samples of the same organisms tested in the absence of effectors.

To determine the presence or absence of an effector compound in a sample a microorganism with known responses is added to the sample with a suitable mediator or mediator mixture. Variation of the microorganism's respiration rate over time is assessed by electrochemical measurement of mediator consumption. This rate is compared with variation of the respiration rate of another portion of the same microorganism not exposed to the test sample.

The mediator or mediator mixture can be any suitable oxidant, including for example one or more of the following: Ferricyanide (hexacyanoferrate(III) is another name for this); Dichlorophenol-indophenol (DCIP); Ferrocene and ferrocene derivatives; Methylene blue; Janus green; Tris(bipyridyl)iron(III); the Quinone class, which includes benzoquinone, naphthoquinone, menadione, anthraquinone, and substituted derivatives of these; and the Phenazine class which includes phenazine methosulfate and phenazine ethosulfate.

In another aspect of the present invention there is provided a method of differentiating Gram-positive and Gram-negative bacteria comprising the steps of: obtaining a test sample of a microorganism; adding a mediator mixture containing a lipid-soluble redox mediator to the test sample; assessing variations of respiration rate of the microorganism over a predetermined time period; and comparing the respiration rate of the microorganism with the respiration rate of another sample of the same microorganism not exposed to the lipid-soluble redox mediator. This rate is compared to variations of the respiratory rate of another sample not exposed to the lipid-soluble mediator compound. The omission of the lipid soluble redox-mediator compound DCIP allows the rapid distinction between cell wall properties commonly used to classify bacteria as Gram-positive and Gram-negative.

The present invention is described in the following examples.

### EXAMPLE 1

Respiratory Cycle Activities in the Presence of Effector Compounds

In this example, the assay uses the following steps: (1) The procedure involves combining 200 μL of the bacterial culture with 1300 μL of buffer (that contains or does not contain the effectors and 5 μM DCIP) for a fixed time (10 min) and at a fixed temperature (35° C.) in a two-electrode electrochemical cell; (2) this is followed by the addition of the mediator (500 μL of 0.2 M potassium ferricyanide) for a fixed time (10 min) and at a fixed temperature (35° C.); and (3) measurement of current at a fixed voltage difference (−100 mV) for a fixed time (120 sec). The ferricyanide solution is added so that the final ferricyanide concentrations is 50 mM.

In this configuration the assay uses two platinum electrodes each having a surface area of approximately 0.017 cm². The measured current is proportional to the concentration of the least-concentrated species of the redox-couple in solution. In the present system, ferricyanide (the oxidized form) is present in great excess, leading microorganisms to produce ferrocyanide (the reduced form) as a result of their respiratory activity. The magnitude of the measured current indicates the quantity of the reduced mediator present in the assay solution. The measured current is integrated over time to produce a plot of total charge against time. The change in total charge is measured between 60 and 120 seconds after the potential is applied.

E. coli B, Enterobacter aerogenes ATCC 13048e, Pseudomonas aeruginosa and Proteus vulgaris ATCC 6380 species were grown at 35° C. over night on commercially available MacConkey agar plates. Bacillus cereus, Eneterococcus faecalis and Staphylococcus aureus ATCC 6538P species were grown at 35° C. overnight on commercially available 5%-sheep blood agar plates. Five to six colonies of each bacterial strain were transferred into a vial containing 5.00 mL buffer solution (pH 7.2) consisting of 4.4 g/L K₂HPO₄, 2.88 g/L KH₂PO₄, 0.48 g/L magnesium sulphate heptahydrate, 0.048 g/L calcium chloride; 1.43 g/L ammonium sulphate and 1.62 g/L ammonium chloride. The bacterial suspensions were adjusted to a MacFarland Standard of 5.0 (5.0×10sup.8 cfu/mL) or an optical density of 1.2 measured at 625 nm and cell growth was stopped by cooling the bacterial suspension for a minimum of 15 min in an ice-bath.

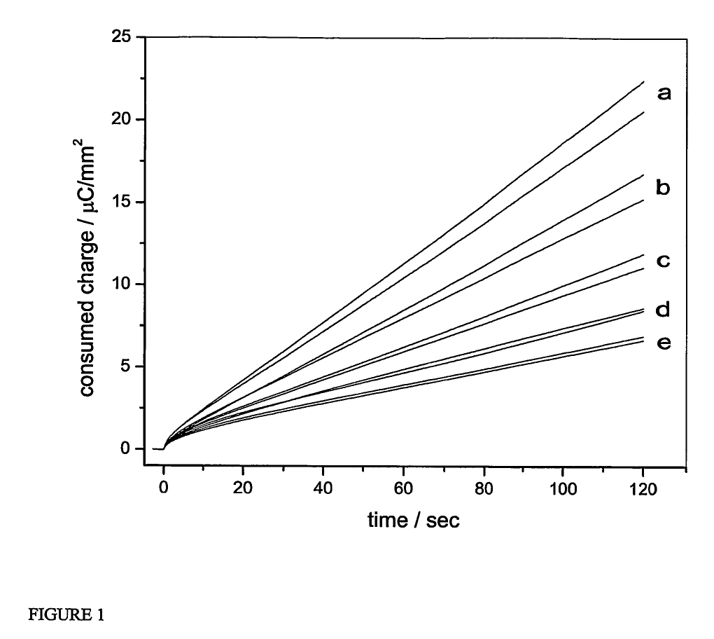

Traces obtained with Enterobacter aerogenes ATCC 13048e incubated in the absence and presence of 10 mM glucose, lactic acid, pyruvic acid, arginine and potassium nitrate are shown in FIG. 1. Table 1 shows the results of these measurements obtained with Enterobacter aerogenes ATCC 13048e and Staphylococcus aureus ATCC 6538P using five different effector compounds. These effectors represent a wide spectrum of known activities and mechanisms of action. Lactose represents the sole carbon source in MacConkey agar plates and consequently cultures grown on MacConkey agar plates possess lactic acid pathways. Pyruvic acid is a metabolic by-product of the tricarboxylic acid cycle of organisms and is needed for the biosynthesis of the amino acids alanine, valine, leucine and glutamate, respectively. In addition, pyruvate can be used for the conversion into glucose (gluconeogenises). Amino acid fermentation (arginine) is carried out by many microbial species. All of the above substrates have been applied to identify and differentiate between bacterial species using existing growth based methods. It is important to note that respiratory cycle activity measurements in the presence of these effector compounds are not the same as results obtained with traditional growth based methods or biochemical screening tests. Instead, respiratory cycle activity measurements are a reflection of the organism's ability to respire under a certain set of conditions.

The results shown in Table 1 have been obtained with Enterobacter aerogenes ATCC 13048e (Gram-negative) and Staphylococcus aureus ATCC 6538P (Gram-positive), which are known to utilize lactic acid as a carbon source. Obtained current signals are integrated, area normalized and relative respiratory cycle activity compared to control measurements (buffer) are plotted for each strain and effector compound.

TABLE 1
Respiration assay results obtained for Staphylococcus aureus
ATCC 6538P and Enterobacter aerogenes ATCC 13048e.

| | | | Staphylococcus aureus ATCC 6538P | | | Enterobacter aerogenes ATCC 13048e | | |
|---|---|---|---|---|---|---|---|---|
| | conc. | | slope | normalized | relative | slope | normalized | relative |
| Effector | mM | n | μC/min | μC/min*mm² | activity | μC/min | μC/min*mm² | activity |
| buffer* | — | 2 | 9.1 | 5.3 | 100% | 16.4 | 9.7 | 100% |
| glucose | 10 | 2 | 17.9 | 10.6 | 198% | 29.6 | 17.5 | 181% |
| lactic A | 10 | 3 | 24.8 | 14.6 | 275% | 27.7 | 16.3 | 169% |
| arginine | 10 | 2 | 9.1 | 5.3 | 100% | 20.5 | 12.1 | 125% |
| pyruvate | 10 | 3 | 14.8 | 8.7 | 164% | 23.4 | 13.7 | 143% |
| nitrate | 10 | 2 | 6.9 | 4.1 | 76% | 3.7 | 2.2 | 23% |

*Respiratory cycle activity in buffer set as 100%-relative activity

Table 2 shows results obtained with Staphylococcus aureus ATCC 6538P and Enterococcus faecalis using two metabolizable effector compounds, glucose and mannose present at 3 different concentrations. The results show an increase in measured respiratory activities as a function of glucose or mannose concentration. It is evident that obtained differences in respiration rates are based on effector concentrations and thus could be used to further aid in the identification and differentiation of microorganism.

TABLE 2
Slope data obtained for S. aureus ATCC 6538P and E. faecalis

| | | S. aureus | | E. faecalis | |
|---|---|---|---|---|---|
| Substrate (n) | Concentration | μC/min | SD | μC/min | SD |
| Buffer (2) | — | 7.8 | ±0.1 | 1.2 | ±0.2 |
| Glucose (2) | 1 mM | 9.3 | ±2.1 | 18.7 | ±0.1 |
| Glucose (2) | 10 mM | 13.9 | ±1.6 | 18.9 | ±0.1 |
| Glucose (2) | 50 mM | 20.1 | ±1.8 | 13.5 | ±0.4 |
| Buffer (2) | — | 10.7 | ±1.5 | 0.8 | ±0.1 |
| Mannose (2) | 1 mM | 16.6 | ±0.1 | 17.8 | ±0.7 |
| Mannose (2) | 10 mM | 14.0 | ±1.2 | 19.6 | ±0.7 |
| Mannose (2) | 50 mM | 15.1 | ±2.1 | 14.5 | ±2.6 |

### EXAMPLE 2

Differentiation Between Gram-Positive and Gram-Negative Bacteria

The following steps are used for each test: (1) preheating for a fixed time (4 min) and a fixed temperature (35° C.) a 150 μL aliquot of buffer containing 1 mM glucose (glc) with or without 5 μM DCIP; (2) this is followed by the addition of 50 μL of the bacterial suspension to the sample for a fixed time (10 min) and at a fixed temperature (35° C.); (3) this is followed by the addition of the mediator (50 μL of 0.4 M potassium ferricyanide) for a fixed time (10 min) and at a fixed temperature (35° C.); and (4) measurement of current at a fixed voltage difference (100 mV) between two platinum electrodes for a fixed time (120 sec) at a fixed temperature (35° C.). The final ferricyanide concentration in the sample prior to reaction with the microorganism is 40 mM.

In this configuration the assay uses two platinum electrodes each having a surface area of approximately 0.03 cm². The measured current depends on the concentration of the least-concentrated species of the redox-couple in solution. In our system, ferricyanide (the oxidized form) is present in great excess, leading microorganisms to produce ferrocyanide (the reduced form) as a result of their respiratory activity. The magnitude of the measured current indicates the quantity of the reduced mediator present in the assay solution. The measured current is integrated over time to produce a plot of total charge against time. The change in total charge is measured between 30 and 120 seconds after the potential is applied.

E. coli B, E. coli Neotype, E. coli HB101, E. coli JM105, Enterobacter aerogenes ATCC 13048e, Pseudomonas aeruginosa and Proteus vulgaris ATCC 6380 species are grown at 35° C. overnight on commercially available MacConkey agar plates. Eneterococcus faecalis and Staphylococcus aureus ATCC 6538P species are grown at 35° C. overnight on commercially available 5%-sheep blood agar plates. Saccharomyces cerevisiae is grown at 35° C. overnight on commercially available Trypton-Soy agar plates. Five to six colonies of each bacterial strain are transferred into a vial containing 3.00 mL buffer solution (pH 6.8) consisting of 4.4 g/L K₂BPO₄, 2.88 g/L KH₂PO₄, 5.00 g/L sodium chloride.

The omission of the lipid soluble redox-mediator compound DCIP allows the rapid distinction between cell wall properties commonly used to classify bacteria as Gram positive and Gram negative. Gram-negative bacteria contain porin proteins (channels) in their outer cell wall allowing ferricyanide to interact directly with terminal components of the respiratory pathway. Gram-positive bacteria do not possess porins and are much less reactive with ferricyanide; full respiratory activity signals are only observed if a lipid-soluble redox mediator, such as DCIP, is present. Table 3 shows a lower respiratory cycle activities obtained for Gram-positive strains after a 10 min incubation period due to the absence of the lipid soluble redox-mediator DCIP in the buffered effector solution.

TABLE 3
Discrimination based on membrane compositions

| | | | | Respiratory | | |
|---|---|---|---|---|---|---|
| | | | | activity | | %-Activity |
| Gram | Species | Condition | N | μC/min | SD± | remaining |
| Positive | E. faecalis | 1 mM glc w/o DCIP | 4 | 7.3 | 0.1 | 15% |
| | | 1 mM glc + DCIP | 4 | 48.2 | 0.5 | |
| Positive | S. aureus | 1 mM glc w/o DCIP | 4 | 13.1 | 0.8 | 39% |
| | | 1 mM glc + DCIP | 4 | 33.8 | 4.0 | |
| Negative | P. vulgaris | 1 mM glc w/o DCIP | 4 | 16.2 | 0.2 | 101% |
| | | 1 mM glc + DCIP | 4 | 16.1 | 6.1 | |
| Negative | E. aerogenes | 1 mM glc w/o DCIP | 4 | 40.1 | 1.7 | 129% |
| | | 1 mM glc + DCIP | 4 | 31.0 | 2.0 | |
| Negative | E. coli B | 1 mM glc w/o DCIP | 4 | 29.0 | 1.1 | 96% |
| | | 1 mM glc + DCIP | 4 | 30.1 | 1.4 | |
| Negative | P. aeruginosa | 1 mM glc w/o DCIP | 4 | 9.2 | 0.2 | 103% |
| | | 1 mM glc + DCIP | 4 | 8.9 | 0.8 | |

Results in Table 3 show that the appropriate choice of redox-mediators and effector compounds allow for the rapid distinction between Gram-positive and Gram-negative bacteria, which further aids in their identification. Similar results have been obtained in the presence of 1 mM mannose or 1 mM succinate (instead of 1 mM glucose), where respiratory signals dropped below 60% and 80% in the absence of 5 μM DCIP, respectively. The appropriate choice of effector compound is necessary to optimize the differences in respiratory activities for the distinction between Gram-positive and Gram-negative bacteria.

### EXAMPLE 3

Differentiation of 10 Microbial Strains Using Pattern Recognition by Principal Component Analysis of Respiratory Activity Data

In this example, each microorganism is subjected to 22 different effector compounds in addition to control measurements. The following steps are used for each test: (1) preheating for a fixed time (4 min) and a fixed temperature (35° C.) a 150 μL aliquot of buffer containing the effector compound and 5 mu.M DCIP; (2) this is followed by the addition of 50 μL of the bacterial suspension to the sample for a fixed time (10 min) and at a fixed temperature (35° C.); (3) this is followed by the addition of the mediator (50 μL of 0.4 M potassium ferricyanide) for a fixed time (10 min) and at a fixed temperature (35° C.); and (4) measurement of current at a fixed voltage difference (100 mV) between two platinum electrodes for a fixed time (120 sec) at a fixed temperature (35° C.). The final ferricyanide concentration in the sample prior to reaction with the microorganism is 40 mM. For each microorganism, duplicate measurements were made of 22 different effector compounds. In addition, duplicate measurements were made a control test containing no effector compound. One additional measurement was made of a control solution containing the microorganism with ferricyanide but without effector or DCIP.

In this configuration the assay uses two platinum electrodes each having a surface area of approximately 0.03 cm.². The measured current depends on the concentration of the least-concentrated species of the redox-couple in solution. In our system, ferricyanide (the oxidized form) is present in great excess, leading microorganisms to produce ferrocyanide (the reduced form) as a result of their respiratory activity. The magnitude of the measured current indicates the quantity of the reduced mediator present in the assay solution. The measured current is integrated over time and normalized for electrode area to produce a plot of total charge against time. The change in total charge is measured between 30 and 120 seconds after the potential is applied.

E. coli B, E. coli Neotype, E. coli HB101, E. coli JM105, Enterobacter aerogenes ATCC 13048e, Pseudomonas aeruginosa and Proteus vulgaris ATCC 6380 species are grown at 35° C. over night on commercially available MacConkey agar plates. Eneterococcus faecalis and Staphylococcus aureus ATCC 6538P species are grown at 35° C. overnight on commercially available 5%-sheep blood agar plates. Saccharomyces cerevisiae is grown at 35° C. overnight on commercially available Trypton-Soy agar plates. Five to six colonies of each bacterial strain are transferred into a vial containing 3.00 mL buffer solution (pH 6.8) consisting of 4.4 g/L K₂HPO₄, 2.88 g/L KH₂PO₄, 5.00 g/L sodium chloride.

In this example, each normalized data set containing duplicate measurements for each of 22 effector compounds is referenced to the duplicate internal controls to allow batch-to-batch comparison. The obtained signals of charge per unit electrode area for the duplicate control measurements are averaged and set at 100% respiratory cycle activity. Signals obtained for each effector compound are then averaged and compared to these control samples and results are displayed as relative %-respiratory activity. The data set used to generate pattern recognition plots consists of 5 replicate batches (each batch containing duplicate measurements for each of 22 effector compounds) for each cell culture (10 strains). This is used to generate a 50 (columns)×22 (rows) matrix. The matrix is imported into MATLAB (Version 6.0). Factor analysis is performed using the MATLAB Chemometric Toolbox (Version 2.3) and involves the generation of reduced eigenvectors to determine the optimal number of factors, examination of the resulting residuals plots for randomness, and the generation of scores for the first four principle components.

TABLE 4
Response classification by species

| | | P. | | E. | E. | E. | | E. | | S. | |
|---|---|---|---|---|---|---|---|---|---|---|---|
| | | vulgaris | E. | aerogenes | coli | coli | E. | coli | P. | aureus | S. |
| Conc. | | ATCC6380 | faecalis | ATCC13048e | JM105 | HB101 | coli B | Neotype | aeruginosa | ATCC6538P | cerevisiae |
| mM | Effector | n = 20 | n = 20 | n = 26 | n = 10 | n = 18 | n = 10 | n = 26 | n = 18 | n = 20 | n = 10 |
| | No effector | 100% | 100% | 100% | 100% | 100% | 100% | 100% | 100% | 100% | 100% |
| | +/−SD | 6% | 18% | 4% | 7% | 6% | 9% | 14% | 6% | 7% | 5% |
| 3 | Succinate | 520% | 87% | 187% | 287% | 172% | 134% | 127% | 102% | 167% | 108% |
| | ′+/−SD | 70% | 11% | +/−24% | +/−47% | 28% | 18% | 27% | 14% | 46% | 8% |
| 10 | L-arginine | 149% | 130% | 129% | 163% | 114% | 137% | 114% | 155% | 132% | 139% |
| | ′+/−SD | 34% | 27% | +/−15% | 19% | 11% | 11% | 21% | 19% | 18% | 12% |
| 20 | D-arabinose | 150% | 139% | 106% | 137% | 107% | 118% | 103% | 113% | 115% | 143% |
| | ′+/−SD | 26% | 50% | 13% | 16% | +/−22% | 15% | 19% | 20% | 25% | 21% |
| 20 | D-xylose | 178% | 128% | 149% | 134% | 110% | 102% | 110% | 113% | 128% | 116% |
| | ′+/−SD | 51% | 26% | 26% | 22% | 18% | 20% | 20% | 17% | 22% | 20% |
| 10 | Beta- | 136% | 120% | 131% | 121% | 110% | 105% | 108% | 114% | 114% | 105% |
| | glycero- | | | | | | | | | | |
| | phosphate | | | | | | | | | | |
| | ′+/−SD | 28% | 32% | 30% | 37% | 13% | 24% | 19% | 21% | 21% | 16% |
| 30 | D-sorbitol | 186% | 179% | 260% | 168% | 124% | 136% | 111% | 104% | 167% | 113% |
| | ′+/−SD | 33% | 33% | 30% | 12% | 29% | 31% | 22% | 13% | 24% | 19% |
| 10 | Pyrovic acid | 344% | 136% | 209% | 249% | 169% | 120% | 126% | 125% | 313% | 109% |
| | ′+/−SD | 101% | 26% | 38% | 21% | 28% | 24% | 30% | 16% | 69% | 11% |
| 20 | D-lactose | 162% | 123% | 268% | 115% | 192% | 164% | 150% | 108% | 138% | 126% |
| | ′+/−SD | 29% | 20% | 44% | 10% | 40% | 21% | 27% | 15% | 25% | 21% |
| 20 | D-fructose | 183% | 463% | 275% | 202% | 202% | 147% | 125% | 136% | 289% | 152% |
| | ′+/−SD | 48% | 126% | 48% | 18% | 39% | 20% | 27% | 39% | 106% | 41% |
| 10 | Formic acid | 222% | 157% | 124% | 136% | 163% | 132% | 114% | 120% | 298% | 132% |
| | ′+/−SD | 60% | 61% | 22% | 34% | 46% | 31% | 24% | 22% | 34% | 14% |
| 20 | Citric acid | 142% | 117% | 163% | 113% | 137% | 161% | 158% | 87% | 83% | 103% |
| | ′+/−SD | 26% | 22% | 49% | 62% | 27% | 37% | 36% | 14% | 11% | 20% |
| 20 | Sucrose | 164% | 465% | 188% | 121% | 110% | 124% | 122% | 117% | 290% | 161% |
| | +/−SD | 47% | 114% | 20% | 10% | 13% | 10% | 21% | 19% | 49% | 16% |
| 10 | L-tryptophan | 521% | 232% | 126% | 133% | 129% | 98% | 93% | 170% | 139% | 188% |
| | ′+/−SD | 125% | 80% | 22% | 20% | 22% | 18% | 19% | 48% | 49% | 28% |
| 10 | Malonic acid | 125% | 146% | 156% | 160% | 124% | 141% | 141% | 125% | 117% | 110% |
| | ′+/−SD | 30% | 33% | 28% | 63% | 24% | 25% | 32% | 26% | 20% | 16% |
| 5 | L-ornithine | 151% | 138% | 142% | 165% | 114% | 135% | 134% | 148% | 154% | 137% |
| | ′+/−SD | 33% | 33% | 17% | 14% | 9% | 30% | 23% | 33% | 22% | 20% |
| 10 | L-lysine | 219% | 243% | 125% | 118% | 125% | 100% | 101% | 186% | 159% | 240% |
| | ′+/−SD | 42% | 46% | 22% | 22% | 13% | 26% | 19% | 27% | 35% | 42% |
| 20 | D-galactose | 341% | 200% | 334% | 173% | 153% | 165% | 155% | 130% | 195% | 143% |
| | +/−SD | 101% | 35% | 63% | 23% | 31% | 43% | 30% | 22% | 55% | 19% |
| 20 | D-mannose | 250% | 654% | 296% | 212% | 222% | 152% | 139% | 108% | 262% | 125% |
| | ′+/−SD | 73% | 168% | 44% | 41% | 38% | 26% | 20% | 14% | 68% | 11% |
| 10 | Alpha- | 201% | 109% | 102% | 142% | 116% | 130% | 100% | 97% | 115% | 124% |
| | ketoglutarate | | | | | | | | | | |
| | ′+/−SD | 46% | 25% | 23% | 18% | 15% | 37% | 25% | 22% | 24% | 17% |
| 10 | Lactic acid | 457% | 118% | 182% | 303% | 171% | 192% | 126% | 122% | 622% | 123% |
| | ′+/−SD | 89% | 33% | 55% | 27% | 40% | 49% | 27% | 27% | 205% | 19% |
| 20 | L-rhaninose | 145% | 105% | 110% | 116% | 103% | 119% | 105% | 94% | 101% | 124% |
| | ′+/−SD | 40% | 24% | 33% | 36% | 19% | 35% | 25% | 20% | 32% | 33% |
| 10 | Beta- | 151% | 155% | 110% | 187% | 132% | 125% | 81% | 90% | 98% | 112% |
| | cyclodextrin | | | | | | | | | | |
| | ′+/−SD | 36% | 57% | 35% | 42% | 46% | 49% | 17% | 21% | 28% | 23% |

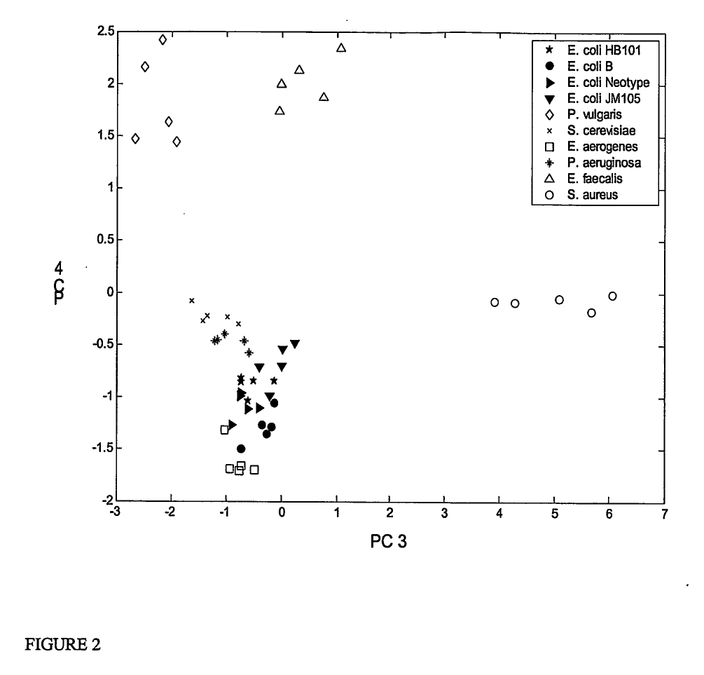

Initially, principal component analysis was applied to %-activity data shown in Table 4, in an attempt to classify responses according to species. Individual measurements (5 replicates of 10 strains) are incorporated as a 22 element column into the matrix, so that each column contained one measurement with each of the 22 effector compounds. Next, all possible combinations of scores for PC 1, PC 2, PC 3 and PC4 were used in two-dimensional plots to examine groupings by species. The resulting scores plot using PC 3 and PC 4 is shown in FIG. 2.

In an effort to optimize obtained pattern recognition results effector ranking followed by rank annihilation was performed. In order to assess the impact of each effector compound, standard deviations of %-respiratory activities are calculated across each row in Table 4. This procedure allows for the ranking of individual effectors to the importance of their contributions to variances found between 10 organisms.

TABLE 5
Effector ranking according to found variances between 10 strains

| Rank | Effector | ±SD |
|---|---|---|
| 1 | Lactic acid | 171% |
| 2 | D-mannose | 158% |
| 3 | Succinate | 130% |
| 4 | L-tryptophan | 126% |
| 5 | Sucrose | 112% |
| 6 | D-fructose | 103% |
| 7 | Pyruvic acid | 86% |
| 8 | D-galactose | 76% |
| 9 | Formic acid | 58% |
| 10 | L-lysine | 56% |
| 11 | D-sorbitol | 48% |
| 12 | D-lactose | 48% |
| 13 | Beta-cyclodextrin | 33% |
| 14 | Alpha-ketoglutarate | 31% |
| 15 | Citric acid | 30% |
| 16 | D-xylose | 23% |
| 17 | D-arabinose | 17% |
| 18 | Malonic acid | 17% |
| 19 | L-arginine | 16% |
| 20 | L-rhamnose | 15% |
| 21 | L-ornithine | 14% |
| 22 | Beta-glycerophosphate | 11% |

Higher standard deviation values for individual effector compounds are considered indicative of the greater importance of that effector for the grouping of organisms in the scores plot. Effectors (rows) are then cumulatively eliminated from the data matrix, beginning with beta-glycerophosphate and continuing upwards from the bottom of Table 5.

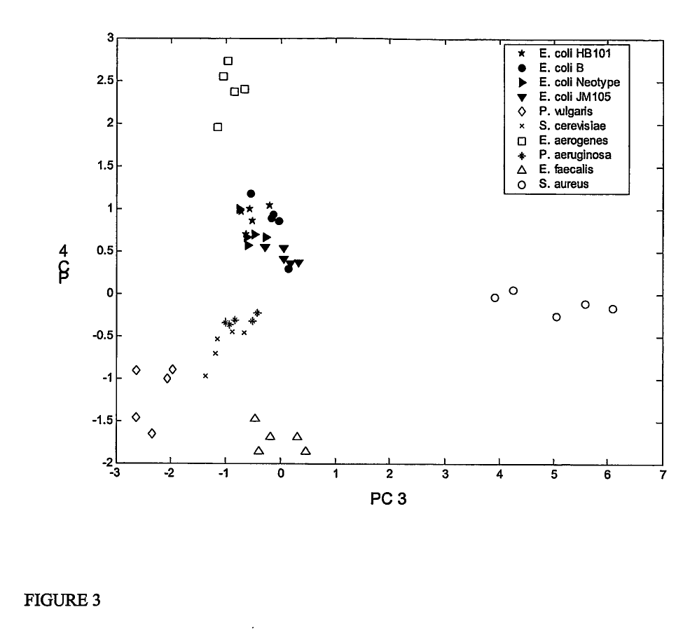

FIG. 3 shows the scores plot of PC 3 and PC 4 obtained from the reduced matrix using the first 16 effector compounds. Additionally, E. coli subspecies (the two wild type strains E. coli B and E. coli Neotype, and the two laboratory strains E. coli JM105 and E. coli HB101) form identifiable subpopulations that are clustered in close proximity to each other with respect to the clusters representing other species.

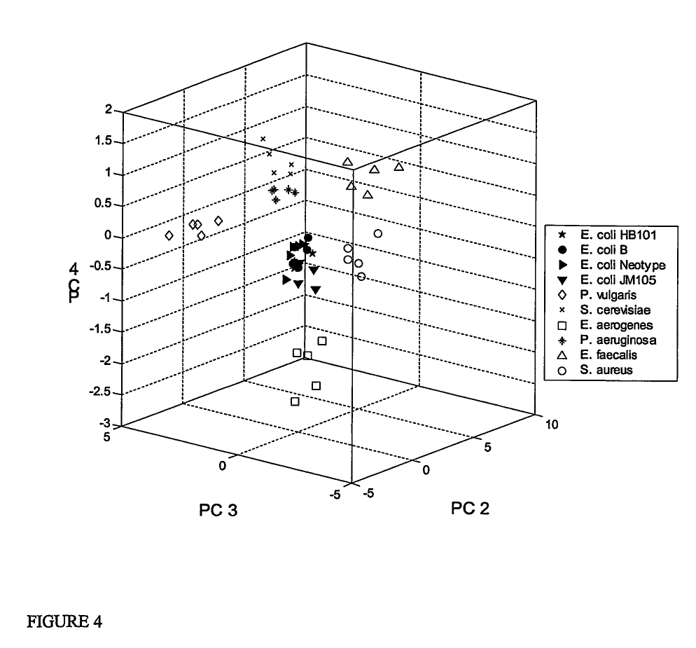

Further reduction of the matrix to 9 effector compounds is possible using a 3-D projection of PC 2, PC3, PC 4 as shown in FIG. 4. This illustrates that the assay method of the present invention can be used for microorganism identification using more complex mathematical methods.

### EXAMPLE 4

Probability Method for the Identification of Unknown Microbial Samples

In this example, experiments were conducted that were identical to Example 3, and data shown in Table 4 were used to establish a reference database for the ten strains of microorganisms. In the reference database, ten individual measurements for each effector compound with each microorganism allow the establishment of a mean respiratory activity value along with its confidence interval. A batch experiment (described in Example 3) conducted on an unknown organism yields two measurements per effector compound. These two values are averaged, and the average is compared to the 90% confidence intervals for the same effector for each organism in the reference database. An “effector match” is indicated for that effector when the measurement for the unknown organism falls within the confidence interval for the same effector compound with one organism in the reference database. The “overall match” is calculated simply as the number of effector matches to a given organism divided by the total number of effectors (twenty-two).

Table 6 shows the results obtained from a comparison of three replicate measurements of 10 unknown samples against the established library. High percentage match values indicate that the unknown culture provides essentially the same pattern of respiratory response to effector compounds as a known sample from the reference database. It can be seen in Table 6 that the highest percentage match values for each of the 30 unknowns are obtained with the database entries corresponding to the same organisms.

These results demonstrate that patterns of electrochemically-measured respiratory activity responses to effector compounds can be used for the identification of unknown microorganisms.

TABLE 6
Identification results obtained using statistical analysis
method by comparison of unknown sample to reference library

| | | % Match | | |
|---|---|---|---|---|
| Unknown | Prediction | 1 | 2 | 3 |
| E. faecalis | E. faecalis | 96% | 100% | 96% |
| | S. cerevisiae | 72% | 76% | 68% |
| | E. coli B | 68% | 68% | 64% |
| E. aerogenes | E. aerogenes | 84% | 92% | 88% |
| | E. coli B | 64% | 76% | 72% |
| | S. aureus | 60% | 60% | 68% |
| P. vulgaris | P. vulgaris | 88% | 88% | 92% |
| | E. coli B | 64% | 60% | 56% |
| | S. aureus | 60% | 48% | 80% |
| S. aureus | S. aureus | 92% | 92% | 88% |
| | E. faecalis | 80% | 84% | 80% |
| | P. vulgaris | 76% | 76% | 76% |
| P. aeruginosa | P. aeruginosa | 96% | 92% | 92% |
| | S. cerevisiae | 88% | 88% | 80% |
| | E. faecalis | 80% | 76% | 80% |
| E. coli B | E. coli B | 88% | 92% | 88% |
| | E. coli Neotype | 88% | 76% | 88% |
| | E. coli HB101 | 84% | 76% | 76% |
| E. coli HB101 | E. coli HB101 | 100% | 84% | 76% |
| | E. coli B | 88% | 84% | 72% |
| | E. coli Neotype | 80% | 60% | 72% |
| E. coli Neotype | E. coli Neotype | 88% | 100% | 96% |
| | E. coli B | 80% | 92% | 92% |
| | E. coli HB101 | 64% | 80% | 84% |
| E. coli JM105 | E. coli JM105 | 92% | 84% | 80% |
| | S. aureus | 80% | 72% | 72% |
| | P. vulgaris | 76% | 64% | 72% |
| S. cerevisisae | S. cerevisiae | 96% | 92% | 88% |
| | P. aeruginosa | 88% | 80% | 88% |
| | E. coli B | 84% | 80% | 80% |

The above examples are illustrative only. It is not intended that the invention be limited to the above examples. Many variations will be apparent to those who are knowledgeable in the field, and such variations are within the scope of the invention as described and claimed, whether or not expressly mentioned herein.

1. A method of identifying a microorganism comprising the steps of:
a) obtaining a test sample of an unknown microorganism;
b) adding a mediator or mediator mixture to the test sample in the presence of an effector;
c) assessing variation in respiration rate of the microorganism over a pre-determined time period; and
d) comparing the variation in the respiration rate of the microorganism with the variation in respiration rates of known microorganisms exposed to the effector, thereby identifying the unknown microorganism in the test sample.

2. The method of claim 1 wherein the step of adding a mediator or mediator mixture to the test sample comprises combining the test sample with a solution of the effector for a fixed time prior to adding the mediator.

3. The method of claim 2 wherein the mediator or mediator mixture comprises an oxidant.

4. The method of claim 3 wherein the mediator or mediator mixture is ferricyanide, dichlorophenol-indophenol (DCIP), ferrocene and ferrocene derivatives, methylene blue, janus green, tris(bipyridyl)iron(III), a quinone, or a phenazine.

5. The method of claim 4 wherein the quinone is benzoquinone, naphthoquinone, menadione, anthraquinone, or substituted derivatives of these.

6. The method of claim 4 wherein the phenazine is phenazine methosulfate or phenazine ethosulfate.

7. The method of claim 1 wherein the respiration rates of the unknown microorganism and known microorganism are assessed using electrochemical measurements.

8. The method of claim 7 wherein the electrochemical measurements are biamperometric or coulometric.

9. The method of claim 7 wherein the respiration rate of the unknown microorganism and the known microorganism are assessed by the electrochemical measurement of mediator consumption.

10. The method of claim 1 wherein the pre-determined time period is up to 15 minutes.

11. The method of claim 1 wherein the unknown microorganism is in an arrested growth state.

12. The method of claim 1 wherein a plurality of effectors are separately employed to assess variations in respiration rate.

13. The method of claim 12 wherein said effector is selected from the group consisting of succinate, D-xylose, D-lactose, ornithine, alpha-ketoglutarate, beta-glycerophosphate, D-fructose, sucrose, L-lysine, lactic acid, L-arginine, D-sorbitol, formic acid, L-tryptophan, D-galactose, L-rhamnose, D-arabinose, pyruvic acid, citric acid, malonic acid, D-mannose, beta-cyclodextrin, nitrate and glucose.

14. A method of differentiating Gram-positive and Gram-negative bacteria comprising the steps of:
a) obtaining a test sample of a bacterium;
b) adding a mediator mixture containing a lipid-soluble redox mediator to the test sample;
c) assessing variations of respiration rate of the bacterium over a pre-determined time period; and
d) comparing the respiration rate of the bacterium with the respiration rate of another sample of the same bacterium not exposed to the lipid-soluble redox mediator, wherein a significant change in respiration rate indicates the presence of a Gram-positive bacterium and no significant change in respiration rate indicates the presence of a Gram-negative bacterium.

15. The method of claim 14 wherein the lipid-soluble redox mediator comprises an oxidant.

16. The method of claim 15 wherein the mediator or mediator mixture is ferricyanide, dichlorophenol-indophenol (DCIP), ferrocene and ferrocene derivatives, methylene blue, janus green, tris(bipyridyl)iron(III), a quinine, or a phenazine.

17. The method of claim 16 wherein the quinone is benzoquinone, naphthoquinone, menadione, anthraquinone, or substituted derivatives of these.

18. The method of claim 16 wherein the phenazine is phenazine methosulfate or phenazine ethosulfate.

19. A method of differentiating individual strains of microorganisms in a plurality of strains of microorganisms, which method comprises the steps of:
a) culturing a plurality of samples of each individual strain of microorganism in the presence of a plurality of effector compounds, each sample being cultured separately with one of the plurality of effector compounds in the presence of a mediator compound;
b) assessing variation in respiration rate of each microorganism in the presence of each effector compound over a pre-determined time period; and
c) transforming data measurements of the variation in respiration rate for each sample of each individual strain of microorganism cultured with an effector compound in the presence of a mediator compound to accentuate differentiating characteristics among said individual strains of microorganisms.

20. The method of claim 19 further comprising ranking individual effector compound efficacy according to the contribution of each individual effector compound to variations in respiration rate among said individual strains of microorganisms.

21. A method of differentiating individual strains of microorganisms in a plurality of strains of microorganisms, which method comprises the steps of:
a) culturing a plurality of samples of each individual strain of microorganism in the presence of a plurality of mediator compounds, each sample being cultured separately with one of the plurality of mediator compounds in the presence of an effector compound;
b) assessing variation in respiration rate of each microorganism in the presence of each mediator compound over a pre-determined time period; and
c) transforming data measurements of the variation in respiration rate for each sample of each individual strain of microorganism cultured with a mediator compound in the presence of an effector compound to accentuate differentiating characteristics among said individual strains of microorganisms.

22. The method of claim 21 further comprising ranking individual mediator compound efficacy according to the contribution of each individual mediator compound to variations in respiration rate among said individual strains of microorganisms.
